(19) United States
(12) Patent Application Publication
(10) Pub. No.: US 2007/0092568 A1
(43) Pub. Date: Apr. 26, 2007

(54) GALANTAMINE COMPOSITIONS

(76) Inventors: Subhash Pandurang Gore, Shaolapur 413 107, Maharashtra (IN); Arun Kant Krishnakumar Rajlakshmy, Thiruvananthapuram, Kerala (IN); Kodipyaka Ravinder, Kaghaznagar 504 296, A.P. (IN); Venkata Nookaraju Sreedharala, Anakapalle 531 001, A.P. (IN); Indu Bhushan, Hyderabad 500 072, A.P. (IN); Mailatur Sivaraman Mohan, Hyderabad 500 072, A.P. (IN)

Correspondence Address: DR. REDDY'S LABORATORIES, INC., 200 SOMERSET CORPORATE BLVD, SEVENTH FLOOR,, BRIDGEWATER, NJ, 08807-2862, US

(21) Appl. No.: 11/539,918

(22) Filed: Oct. 10, 2006

Related U.S. Application Data
(60) Provisional application No. 60/745,243, filed on Apr. 20, 2006.

(30) Foreign Application Priority Data
Oct. 10, 2005 (IN) 1447/CHE/2005

(51) Int. Cl. A61K 9/24; A61K 31/55

(52) U.S. Cl. 424/472; 514/214.01

(57) ABSTRACT

The present invention relates to controlled release compositions of galantamine, processes to prepare the compositions, their in-vitro release profiles and method of use and method of treatment using the said compositions.

### INTRODUCTION TO THE INVENTION

The present invention relates to controlled release compositions of galantamine, processes to prepare the compositions, their in vitro release profiles and methods of use and methods of treatment using the said compositions.

Galantamine is chemically known as (4aS,6R,8aS)-4a,5,9,10,11,12-hexahydro-3-methoxy-11-methyl-6H-benzofuro[3a,3,2-ef][2]benzazepin-6-ol and is structurally represented as Formula I. It is a reversible inhibitor of acetylcholinesterase that binds specifically to the nicotinic receptors. Galantamine hydrobromide is a white to almost white powder and is sparingly soluble in water.

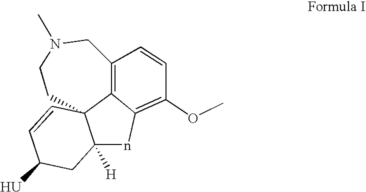

Galantamine hydrobromide been approved in the United States for treating Alzheimer's disease. It is marketed by Janssen under the brand name of RAZADYNE™ tablets containing galantamine hydrobromide and having strengths of 4, 8, and 12 mg; RAZADYNE™ oral solution of strength 4 mg/ml; and RAZADYNE™ ER extended release capsules of strengths 8, 16, and 24 mg. These strengths are expressed as contained galantamine base. RAZADYNE™ is indicated for the treatment of mild to moderate dementia of the Alzheimer's type.

International Application Publication Nos. WO 00/38686 and WO 2005/048979 disclose modified release formulations containing galantamine.

International Application Publication No. WO 2005/065661 discloses a fast dissolving formulation and a sustained release formulation comprising galantamine.

U.S. Patent Application Publication No. 2004/0097484 discloses a once-daily pharmaceutical composition comprising galantamine.

The extended release pharmaceutical composition of the present invention exhibits a desired in vitro dissolution profile and can serve as an economical alternative to the marketed product, RAZADYNE™ ER extended release capsules, with a decrease in the frequency of administration and thus, better patient compliance.

This and other needs are addressed by the present invention.

### SUMMARY OF THE INVENTION

An aspect of present invention provides for a pharmaceutical composition comprising galantamine or a salt thereof, a portion of the contained galantamine being present in an immediate release form and another portion being present in an extended release form.

Another aspect of present invention provides for a pharmaceutical composition comprising galantamine or a salt thereof in combination with a rate controlling substance, having an exterior coating of the same or a different rate controlling substance.

In one aspect, a pharmaceutical composition comprises a capsule containing tablets comprising galantamine or a salt thereof, the tablets being coated with a rate controlling substance.

In another aspect, a pharmaceutical composition comprises a capsule containing pharmacologically inert particles having a coating comprising galantamine or a salt thereof and a rate controlling substance, and having an exterior coating comprising a rate controlling substance.

In an embodiment of the invention, a pharmaceutical composition comprises a portion of the contained galantamine, which is present in more than one extended release form.

In another embodiment of the invention, an immediate release form of pharmaceutical composition comprises pharmacologically inert particles that are coated with galantamine or a salt thereof.

In further embodiment of the invention an extended release form of pharmaceutical composition comprises pharmacologically inert particles that are coated with galantamine or a salt thereof, and having an outer coating comprising a rate controlling substance.

In an embodiment of the invention, controlled release compositions of galantamine provides a release of at least about 30 to about 70 percent of contained galantamine in about 1 hour and more than about 70 percent of the galantamine in about 10 hours, as measured in a buffer pH 6.8 at 37° C., using USP dissolution apparatus 2 at 50 rpm.

An embodiment of the invention includes a pharmaceutical composition comprising galantamine or a salt thereof, a portion of the contained galantamine being present in an immediate release form and another portion being present in an extended release form.

Another embodiment of the invention includes a pharmaceutical composition comprising galantamine or a salt thereof in combination with a rate controlling substance, having an exterior coating of the same or a different rate controlling substance.

A further embodiment of the invention includes a pharmaceutical composition comprising a capsule containing particles comprising galantamine or a salt thereof, the particles being coated with a rate controlling substance.

### DETAILED DESCRIPTION

The term “active ingredient” herein refers to a pharmaceutically active molecule as well as its pharmaceutically acceptable and therapeutically active salts, esters, amides, prodrugs, metabolites, enantiomers, polymorphs, analogs, etc. that induce a desired pharmacological or physiological effect. Terms like “active”, “active agent”, “active substance”, “active pharmaceutical substance”, “pharmacologically active agent”, “drug” and “drug substance” may be used synonymously for “active ingredient”.

The present invention relates to controlled release compositions of galantamine, processes to prepare the compositions, their in vitro release profiles and methods of use and methods of treatment using the compositions.

Different aspects of the invention include, without limitation thereto:

capsule compositions comprising mini-formulations in the form of pellets;

capsule compositions comprising mini-formulations in the form of compressed tablets;

capsule compositions comprising mini-formulations in the form of pellets and compressed tablets;

mini-formulations having cores comprising an active ingredient and water-soluble or water-insoluble components, coated with combinations of at least a water-soluble and at least a water-insoluble component;

mini-formulations having cores comprising an active ingredient and coated with a combination of at least a water-soluble and at least a water-insoluble component;

mini-formulations having a core coated with a mixture of active ingredient and a water-soluble or water-insoluble component and further coated with combination of at least a water-soluble and at least a water-insoluble component; and

mini-formulations having a core coated with active ingredient and further coated with a combination of at least a water-soluble and at least a water-insoluble component.

In an embodiment, the present invention includes controlled release compositions of galantamine, wherein compositions comprise two separate portions wherein one portion releases galantamine in an immediate release (“IR”) manner and other portion releases galantamine in an extended release (“ER”) manner.

In another embodiment of the invention, controlled release compositions of galantamine comprise galantamine and at least one rate controlling substance in a single layer with or without other pharmaceutically acceptable excipients.

In an embodiment of the invention, controlled release compositions of galantamine provides a release of at least about 30% to about 70% of galantamine in about 1 hour and more than about 70% of galantamine in about 10 hours, as measured in a buffer pH 6.8 at 37° C., using USP dissolution apparatus 2 at 50 rpm.

Galantamine used in the present invention can be in the form of the base or a pharmaceutically acceptable salt, or combinations of base and one or more salts, or combinations of one or more salts. Pharmaceutically acceptable salts of galantamine include but are not limited to the hydrochloride, hydrobromide and the like.

In an embodiment of the invention, galantamine hydrobromide is a useful active ingredient in the range of about 2 mg to 60 mg, or about 4 mg to 40 mg, per dosing unit.

In an aspect of the invention, compositions comprising two separate portions may be presented in the form of particulate compositions comprising immediate release galantamine particles and extended release galantamine particles in a defined ratio either filled into a capsule shell or compressed as a tablet formulation or filled into sachets.

In an embodiment of the invention, the ratio of the two portions IR to ER may range from 10:90 to 50:50, or 20:80 to 30:70, w/w equivalent to total galantamine present in the dosage form.

Immediate release particles of the invention may be prepared as powders, granules, pellets, beads and the like using manufacturing processes such as direct blending, dry granulation, wet granulation, pelletization techniques such as but not limited to extrusion-spheronization, dry powder or solution or dispersion layering of galantamine onto inert beads or pellets or particles using conventional coating techniques or fluid bed coating techniques.

Extended release particles of the invention may be prepared as powders, granules, pellets, beads and the like using manufacturing processes such as direct blending, dry granulation, wet granulation, pelletization techniques such as but not limited to extrusion-spheronization, dry powder or solution or dispersion layering of galantamine onto inert beads or pellets or particles using conventional coating techniques or fluid bed coating techniques. Extended release particles may comprise galantamine and rate controlling substance together in one layer, or galantamine and a portion of rate controlling substance together in one layer and a remaining portion of rate controlling substance in a different layer, or galantamine and rate controlling substances together in different layers. In a specific embodiment of the invention wherein galantamine and rate controlling substances are together in different layers, then a galantamine layer comprises a water insoluble component.

In an embodiment of the invention, two portions of the composition comprise:

1) IR portion comprising
a) Galantamine hydrobromide and water insoluble component loaded onto inert particles such as Celphere.
b) Drug loaded pellets are optionally coated with a film coating substance.

2) ER portion comprising
a) Galantamine hydrobromide and a water insoluble component loaded onto inert particles such as Celphere.
b) Drug loaded particles are coated with rate controlling substance with or without additional pharmaceutically acceptable excipients.
c) Above coated pellets are further optionally coated with a film coating substance.
d) Defined ratio of particles from a) and b) are blended with or without other pharmaceutically acceptable excipients.
e) Above blend is either filled into a capsule or compressed as a tablet. Tablets are further optionally coated.

In another embodiment of the invention, wherein galantamine and at least one rate controlling substance are present in a single layer include but not limited to compositions:
a) Particulate compositions of galantamine and at least one rate controlling substance are mixed or dry or wet granulated or extruded-spheronized and said particles are further optionally coated with a film coating substance or same or different rate controlling substances.
b) Particulate compositions wherein galantamine and at least one rate controlling substance together are layered onto an inert particle or bead or pellet through powder or solution or dispersion coating using conventional or fluid bed coating systems and said particles are further optionally coated with a film coating substance or same or different rate controlling substances.
c) In the above two options, the galantamine portion may either contain a water-soluble or water insoluble component.

Useful water-soluble components include, but are not limited to: cellulosic polymers such as carboxymethyl cellulose, hydroxypropyl methylcellulose and hydroxypropyl cellulose; polyethylene oxide; polyvinyl alcohol; carbomer; carageenan; sugars such as mannitol and lactose; and mixtures thereof.

Useful water-insoluble components include, but are not limited to: acrylic acid derivatives; cellulose polymers including alkyl derivatives of cellulose like ethylcellulose, cellulose esters such as cellulose acetate, cellulose propionate, cellulose acetate propionate, cellulose acetate butyrate, cellulose acetate phthalate, and cellulose triacetate; waxes such as beeswax, carnauba wax, and microcrystalline wax; fatty alcohols such as cetostearyl alcohol, stearyl alcohol, cetyl alcohol, and myristyl alcohol; and fatty acid esters like glyceryl monostearate, glycerol monooleate, acetylated monoglycerides, tristearin, tripalmitin, cetyl esters wax, and glyceryl palmitostearate, glyceryl behenate; and hydrogenated castor oil.

In an embodiment of the invention, ethylcellulose and various grades of Eudragit™ products such as Eudragit NE30D were found to be useful as a water-insoluble component. Eudragit NE30D is a 30% aqueous dispersion of poly(ethylacrylate-methylmethactylate).

Ethylcellulose is commercially available as Ethocel®. Ethocel® Premium is available in different viscosities like 7 cps, 10 cps, 20 cps, 45 cps and 100 cps, with an average particle size more than 250 μm. It is used by dissolving in an organic solvent for the preparation of dosage form while Ethocel® standard FP premium which is available in viscosities 7 cps, 10 cps and 100 cps is very finely milled and can be used for direct compression in matrix compositions. Ethylcellulose is also available as an aqueous dispersion under the trade name of Aquacoat® ECD, Aqualon® and Surelease®.

Surelease® is a plasticized aqueous dispersion of ethylcellulose used for extended release coatings and taste masking applications, available in 25% by weight solid content and manufactured by Colorcon Ltd. of Dartford Kent, United Kingdom.

In another aspect of the invention, the water-soluble component is used as a pore-forming agent. The term “pore-forming agent” refers to a pharmaceutically acceptable agent that dissolves in its surrounding medium and results in formation of pores in the membrane to facilitate the diffusion of active ingredient through the membrane.

In one aspect of the invention, hydroxypropyl methylcellulose was found to be useful as a water-soluble component.

Compositions of present invention may comprise pharmaceutical excipients such as, but not limited to, diluents, binders, disintegrants, colourants, anti-oxidants, sweeteners, and film-forming agents.

Common diluents useful in the present invention include, but are not limited to, microcrystalline cellulose, silicified microcrystalline cellulose, microfine cellulose, lactose, starch, pregelatinized starch, calcium carbonate, calcium sulfate, sugar, dextrates, dextrin, dextrose, mannitol, sorbitol, dibasic calcium phosphate dihydrate, tribasic calcium phosphate, kaolin, magnesium carbonate, magnesium oxide, maltodextrin, polymethacrylates, and mixtures thereof.

Binders useful in the present invention include, but are not limited to, starches, microcrystalline cellulose, methylcellulose, cellulose ethers, sodium carboxymethylcellulose, ethylcellulose, dextrose, lactose, sucrose, sorbitol, mannitol, polyethylene glycol, polyvinylpyrrolidone, pectins, gelatin, polyacrylamides, polyvinyloxoazolidone, polyvinylalcohols, and mixtures thereof.

Inert beads or pellets useful in the present invention include, but are not limited to microcrystalline cellulose spheres, silicon dioxide or glass beads, dicalcium phosphate particles, plastic (polypropylene or polyethylene) resin particles, and the like.

In an embodiment of the invention, Celphere™ microcrystalline cellulose spheres manufactured by Asahi Kasei Chemicals Corporation, Tokyo, Japan are useful.

Rate controlling substances useful in the present invention include but are not limited to water soluble or water insoluble substances. Water soluble rate controlling substances include but are not limited to cellulose derivatives such as methylcellulose, carboxymethyl cellulose, hydroxypropyl methylcellulose, cross-linked sodium carboxymethyl cellulose, and cross-linked hydroxypropyl cellulose; carboxymethylamide; potassium methacrylate/divinylbenzene copolymers; polymethylmethacrylate; polyhydroxyalkyl methacrylate; cross-1 inked polyvinylpyrrolidone; high-molecular weight polyvinylalcohols; gums such as natural gum, agar, agrose, sodium alginate, carrageenan, fucoidan, furcellaran, laminaran, hypnea, eucheums, gum arabic, gum ghatti, gum karaya, gum tragacanth and locust bean gum; hydrophilic colloids such as alginates, carbopol and polyacrylamides; other substances such as arbinoglactan, pectin, amylopectin, gelatin, N-vinyl lactams, polysaccharides; and the like. Combinations of any two or more of these polymers, and other polymers having the required properties are within the scope of the invention.

Water insoluble substances include but not limited to celluloses such as methyl cellulose, ethyl cellulose, low-substituted hydroxypropylcellulose (L-HPC), cellulose acetates and their derivatives, cellulose acetate phthalate, hydroxypropyl methylcellulose phthalate, cellulose acylate, cellulose diacylate, cellulose triacylate, cellulose acetate, cellulose diacetate, cellulose triacetate, mono-, di- and tri-cellulose alkanylates, mono-, di-, and tri-cellulose arylates, and mono-, di- and tri-cellulose alkenylates, crosslinked vinylpyrrolidone polymers (also called “crospovidone”), glyceryl behenate, polymethacrylic acid based polymers and copolymers sold under the trade name of EUDRAGIT™ (including Eudragit RL and RS, NE-30D), zein, and aliphatic polyesters. Other classes of polymers, copolymers of these polymers or their mixtures in various ratios and proportions as required are within the scope of this invention without limitation.

The composition disclosed in the present invention is useful in clinical conditions requiring administration of galantamine and pharmaceutically acceptable salts. Such conditions include treatment of Alzheimer's dementia and related dementias, facial neuralgia, alcoholism, nicotine dependence, nerve gas poisoning, mania, chronic fatigue syndrome, schizophrenia, and negative effects of benzodiazepine treatment.

The following examples further illustrate certain specific aspects and embodiments of the invention in greater detail and are not intended to limit the scope of the invention.

### EXAMPLE 1

### Galantamine ER Capsules (8 mg) Comprising Two Particulate Compositions

| | |
|---|---|
| | Quantity/Batch |
| Ingredients | (g) |

Immediate Release Pellets

| | |
|---|---|
| Microcrystalline cellulose spheres | 308 |
| (Celphere CP507)* | |
| Galantamine HBr | 42 |
| Eudragit NE30D | 10 |
| Isopropyl alcohol | 786.2 |
| Water | 461.8 |

Extended Release Pellets

| | |
|---|---|
| Celphere CP507 | 1234 |
| Galantamine HBr | 164 |
| Eudragit NE30D** | 42 |
| Isopropyl alcohol | 3115 |
| Water | 1829 |

ER coating (10% w/w)

| | |
|---|---|
| Ethyl cellulose | 92 |
| Eudragit EPO** | 4 |
| Acetyltributyl citrate | 10 |
| Talc | 38 |
| Isopropyl alcohol | 1692 |
| Water | 564 |

*Celphere ™ CP507 (microcrystalline cellulose spheres, particle size range 500-710 μm) is manufactured by Asahi Kasei Chemicals Corporation, Tokyo, Japan.
**Eudragit NE30D and Eudragit EPO are manufactured by Rohm and Haas.

Manufacturing Process:
Immediate Release Pellets:
1. Galantamine hydrobromide was dispersed in isopropyl alcohol with stirring.
2. Water was slowly added to the dispersion of step 1 with stirring until a clear solution was obtained.
3. Eudragit NE30D was added to the solution of step 2 with stirring at 2500 rpm
4. The solution of step 3 was spray coated over Celphere™ pellets in a fluidized bed processor (Gansons Limited, India).
5. Coated pellets of step 4 were dried to achieve loss on drying less than about 2% w/w determined at 105° C.
   Extended Release Pellets:
1. Galantamine hydrobromide was dispersed in isopropyl alcohol with stirring.
2. Water was slowly added to the dispersion of step 1 with stirring until a clear solution was obtained.
3. Eudragit NE30D was added to the solution of step 2 with stirring at 2500 rpm.
4. The solution of step 3 was spray coated over Celphere™ in a fluidized bed processor (Gansons Limited, India).
5. Coated pellets of step 4 were dried to achieve loss on drying less than about 2% w/w determined at 105° C.
6. Eudragit EPO was dissolved in isopropyl alcohol and ethyl cellulose and acetyltributyl citrate were added with stirring to the clear solution.
7. Talc was dispersed in water under stirring.
8. Talc dispersion was added to a solution of step 6.
9. The drug-coated pellets of step 5 were further coated with the dispersion of step
8 till 10% weight built up was obtained.
10. Coated pellets of step 9 were dried to achieve loss on drying less than about 2% w/w determined at 105° C.
11. The immediate release pellets and the extended release pellets were mixed and filled into a capsule.

In vitro Release Profile with the Following Parameters:
Media: Phosphate buffer pH 6.8.
Volume: 500 ml

Apparatus 2 (Paddle) from Test 711-Dissolution in United States Pharmacopeia 24, United States Pharmacopeial Convention, Inc., Rockville, Md. (1999).

Speed: 50 rpm

| Time (hours) | Percent Drug Released |
|---|---|
| 0 | 0 |
| 1 | 27 |
| 2 | 42 |
| 4 | 61 |
| 8 | 80 |
| 12 | 88 |

### EXAMPLE 2

### Galantamine Capsules Comprising Two Particulate Compositions

| | |
|---|---|
| Ingredients | Quantity/Batch (g) |

Drug Loading

| | |
|---|---|
| Celphere CP507 | 7.7 |
| Galantamine HBr | 1.2 |
| Eudragit NE30D | 0.3 |
| Isopropyl alcohol | 23.9 |
| Water | 14.1 |

ER coating

| | |
|---|---|
| Ethyl cellulose | 1.1 |
| Eudragit EPO | 0.06 |
| Acetyltributyl citrate | 0.12 |
| Talc | 0.47 |
| Isopropyl alcohol | 20.6 |
| Water | 6.9 |

Manufacturing Process:

A. Drug Loading:
1. Galantamine HBr was dispersed in isopropyl alcohol with stirring.
2. Water was slowly added to the dispersion of step 1 with stirring until a clear solution was obtained.
3. Eudragit NE30D was added to the solution of step 2 with stirring at 2500 rpm
4. The solution of step 3 was spray coated over Celphere™ in a fluidized bed processor (Gansons Limited, India).
B. Extended Release Coating Solution:
5. Eudragit EPO was dissolved in isopropyl alcohol and ethylcellulose and acetyltributyl citrate were added with stirring to the clear solution.
6. Talc was dispersed in water under stirring.
7. Remaining quantity of water was added to the step 5 solution and stirred until a clear solution was formed.
8. Dispersion of step 6 was added to clear solution of step 7 under stirring.
9. The drug-coated pellets of step 4 were further coated with the suspension of step 8 to get a weight build up of 6% w/w.
10. ER pellets and IR pellets from Example 1 (65 parts and 35 parts by weight, respectively) were blended and filled into capsules.

In Vitro Release Profile with the Following Parameters:
Media: Phosphate buffer pH 6.8.
Volume: 500 ml

Apparatus: USP apparatus 2 (Paddle) from Test 711-Dissolution in United States Pharmacopeia 24, United States Pharmacopeial Convention, Inc., Rockville, Md. (1999).

Speed: 50 rpm

| Time (hours) | Percent Drug Released |
|---|---|
| 0 | 0 |
| 1 | 44 |
| 2 | 58 |
| 4 | 75 |
| 8 | 92 |
| 12 | 99 |

### EXAMPLE 3

### Capsules Comprising Galantamine and Rate Controlling Substance in One Layer Coated Over Inert Particle

| Ingredients | Quantity/Batch (g) |
|---|---|
| Celphere CP507 | 284.8 |
| Galantamine HBr | 102.5 |
| Ethyl cellulose 7 cps | 512.6 |
| Isopropyl alcohol | 7013 |
| Water | 4675 |

Manufacturing Process:
1. Galantamine HBr was dispersed in isopropyl alcohol with stirring.
2. Water was slowly added to the dispersion of step 1 with stirring until a clear solution was obtained.
3. Ethyl cellulose was added to the solution of step 2 with stirring at 2500 rpm
4. The solution of step 3 was spray coated over Celphere™ in a fluidized bed processor (Gansons Limited, India).
5. The coated pellets were filled into a capsule.

### EXAMPLE 4

### Capsules Comprising Galantamine and Rate Controlling Substance

| | |
|---|---|
| Ingredients | Quantity/Batch (g) |

Dry mix

| | |
|---|---|
| Microcrystalline cellulose (“MCC”) | 1594.9 |
| PH101 | |
| Galantamine HBr | 205.1 |
| Water | 1200 |

ER coating

| | |
|---|---|
| Ethyl cellulose | 95 |
| Eudragit EPO | 5 |
| Acetyltributyl citrate | 40 |
| Talc | 10 |
| Isopropyl alcohol | 1762.5 |
| Water | 588 |

Manufacturing Process:
A. Core Pellets
1. Mixed galantamine and MCC PH101 in rapid mixer granulator for 10 minutes followed by granulation with water until a suitable wet mass was observed.
2. The wet mass was extruded using 1.0 mm sieves followed by spheronization at 750 rpm for 10 minutes until uniform wet pellets were formed.
3. The pellets were dried at 60° C. in a fluid bed dryer until the loss on drying (“LOD”) at 105° C. was below 1.5%.
   B. Extended Release Coating
4. Eudragit EPO was dissolved in isopropyl alcohol and ethyl cellulose and acetyltributyl citrate were added with stirring to the clear solution.
5. Talc was dispersed in water under stirring.
6. Remaining quantity of water was added to step 4 and stirred until a clear solution was formed.
7. Dispersion of step-5 was added to the clear solution of step 6 under stirring.
8. The core pellets of step 3 were further coated with the suspension of step 7 in a fluidized bed processor (Gansons Limited, India) to get a weight build up of 6% w/w.
9. The coated pellets were filled into a capsule.

### EXAMPLE 5

### Capsules Comprising Galantamine and Different Amounts of Rate Controlling Substance

| | |
|---|---|
| Ingredients | Quantity/Batch (g) |

Dry mix

| | |
|---|---|
| MCC PH101 | 1594.9 |
| Galantamine HBr | 205.1 |
| Water | 1200 |

ER coating (Pellets A)

| | |
|---|---|
| Ethyl cellulose | 95 |
| Eudragit EPO | 5 |
| Acetyl tributyl citrate | 40 |
| Talc | 10 |
| Isopropyl alcohol | 1762.5 |
| Water | 588 |

ER coating (Pellets B)

| | |
|---|---|
| Ethyl cellulose | 95 |
| Eudragit EPO | 5 |
| Acetyltributyl citrate | 40 |
| Talc | 10 |
| Isopropyl alcohol | 1762.5 |
| Water | 588 |

Manufacturing Process:
A. Core Pellets
1. Mixed galantamine and microcrystalline cellulose in rapid mixer granulator (Sans 20L) for 10 minutes followed by granulation with water until a suitable wet mass was observed.
2. The wet mass was extruded using 1.0 mm sieves followed by spheronization at 750 rpm for 10 minutes until uniform wet pellets were formed.
3. The pellets were dried at 60° C. in a fluid bed dryer until the LOD at 105° C. was below 1.5% w/w.
   B. Extended Release Coatings
4. Eudragit EPO was dissolved in isopropyl alcohol and ethylcellulose and acetyltributyl citrate were added with stirring to the clear solution.
5. Talc was dispersed in water under stirring.
6. Remaining quantity of water was added to the step 5 and stirred until a clear solution was formed.
7. Dispersion of step 5 was added to clear solution of step 6 under stirring.
8. The core pellets of step 3 were further coated with the suspension of step 7 in a fluidized bed processor (Gansons Limited, India) until weight build ups of 3% w/w (Pellets A) and 6% w/w (pellets B) were obtained.
9. Mixed Pellets A and Pellets B in the ratio of 1:1 by weight.
10. The mixed pellets were filled into a capsule.

### EXAMPLE 6

### Capsules Comprising Galantamine and Different Amounts of Rate Controlling Substance

| | |
|---|---|
| Ingredients | Quantity/Batch (g) |

Dry mix

| | |
|---|---|
| MCC PH101 | 1594.9 |
| Galantamine HBr | 205.1 |
| Water | 1200 |

ER coating (Pellets A)

| | |
|---|---|
| Ethyl cellulose | 82 |
| Acetyltributyl citrate | 8 |
| Isopropyl alcohol | 607.5 |
| Water | 202.5 |

ER coating (Pellets B)

| | |
|---|---|
| Eudragit NE 30 D | 82 |
| Acetyltributyl citrate | 8 |
| Isopropyl alcohol | 202.5 |
| Water | 607.5 |

Manufacturing Process:
A. Core Pellets
1. Mixed galantamine and MCC PH101 in rapid mixer granulator for 10 minutes followed by granulation with water until a suitable wet mass was observed.
2. The wet mass was extrudated using 1.0 mm sieves followed by spheronization at 750 rpm for 10 minutes until uniform wet pellets are formed.
3. The pellets were dried at 60° C. in fluid bed dryer until the LOD at 105° C. was below 1.5% w/w.
   B. Extended Release Coating Solution (for Pellets A):
4. Ethyl cellulose was dissolved in isopropyl alcohol with stirring and water was added until a clear solution was obtained.
5. Acetyltributyl citrate was added to the step 4 solution under stirring.
6. The part of core pellets of step 3 were further coated with the solution of step 5 in a fluidized bed processor until a weight build up of 3% w/w was obtained (Pellets A).
   C. Extended Release Coating Solution (for Pellets B):
7. Eudragit NE 30D was dispersed in water with stirring.
8. Acetyltributyl citrate was dissolved in isopropyl alcohol and added to the step 7 dispersion under stirring.
9. The second part of core pellets of step 3 were further coated with the dispersion of step 8 in a fluidized bed processor (Gansons Limited, India) until a weight build up of 6% w/w was obtained (Pellets B).
10. Mixed Pellets A and Pellets B in the ratio of 30:70 by weight.
11. The mixed pellets were filled into a capsule.

### EXAMPLE 7

### Capsules Comprising Mini-Formulations as Compressed Dosage Forms

| | |
|---|---|
| Ingredients | Quantity/Batch (g) |

Core

| | |
|---|---|
| Galantamine hydrobromide | 153.8 |
| Microcrystalline cellulose | 1061.3 |
| Povidone K-90 | 75 |
| Magnesium stearate | 30 |
| Water | 425 |

Coating

| | |
|---|---|
| Ethyl cellulose | 118.8 |
| Hydroxypropyl methylcellulose | 13.2 |
| Isopropyl alcohol | 1188 |

Manufacturing Process:
A. Preparation of Mini-Formulations:
1. Galantamine hydrobromide and microcrystalline cellulose were passed through an ASTM 40 mesh sieve and blended in a planetary mixer.
2. Povidone was dissolved in water with stirring.
3. Blend of step 1 was granulated using the povidone solution of step 2.
4. The granules were dried in a fluidized bed dryer at a temperature 60° C., until the loss on drying was below 1% (measured using a halogen moisture balance at 105° C.)
5. The granules of step 4 were passed through an ASTM 25 mesh sieve.
6. The oversized granules were milled in a comminuting mill and passed through an ASTM 25 mesh sieve.
7. Magnesium stearate was passed through an ASTM 40 mesh sieve.
8. The combined granules of steps 5 and 6 were blended with the magnesium stearate of step 7.
9. The blend of step 8 was compressed in a rotary tablet-punching machine using standard concave punches of diameter 2.5 mm.
   B. Coating of Mini-Formulations:
1. Ethylcellulose and hydroxypropyl methylcellulose were dissolved in isopropyl alcohol.
2. Compressed mini-formulations prepared in Part A were coated with the solution of step 1 in a perforated coating pan (Gans coater) and dried at 45° C.
   C. Filling Tablets into a Capsule: about 11 Coated Mini-Formulations (Equivalent to 8 mg Galantamine Base) Prepared in Part B were Incorporated into a Size 1 Capsule.

### EXAMPLE 8

### Capsules Containing Mini-Formulations in the Form of Pellets

| | |
|---|---|
| Ingredients | Quantity/Batch (g) |

Core components

| | |
|---|---|
| Galantamine hydrobromide | 102.5 |
| Ethyl cellulose (7 cps) | 102.5 |
| Microcrystalline cellulose spheres | 695 |
| (Celphere CP507) | |
| Isopropyl alcohol | 2337 |
| Water | 1558 |

Coating components

| | |
|---|---|
| Surelease ® | 316.8 |
| Hydroxypropyl methylcellulose (5 cps) | 8.8 |
| Water | 50 |

Manufacturing Process:
1. Galantamine hydrobromide was dispersed in isopropyl alcohol with stirring.
2. Water was slowly added to the dispersion of step 1 with stirring until a clear solution was obtained.
3. Ethylcellulose was dissolved in the solution of step 2 with stirring at 2500 rpm
4. The solution of step 3 was spray coated over Celphere™ in a fluidized bed dryer (Gansons Limited, India).
5. Hydroxypropyl methylcellulose was dissolved in water and Surelease® was added with stirring.
6. The drug-coated pellets of step 4 were further coated with the suspension of step 5.
7. Coated pellets were filled into a capsule.

### EXAMPLE 9

### In-Vitro Dissolution of Capsules Containing Galantamine Mini-Formulations

Dissolution conditions (Test 711-Dissolution from United States Pharmacopeia 24, United States Pharmacopoeial Convention, Inc., Rockville, Md., 1999).
Dissolution medium: Phosphate buffer pH 6.8
Volume of dissolution medium: 500 ml
Dissolution apparatus: Apparatus 1 (Basket type)

Stirring speed 100 rpm

| | |
|---|---|
| Time | Percent Drug Released |

| (hours) | Example 7 | Example 8 |
|---|---|---|
| 1 | 29.5 | 25 |
| 2 | 77.5 | 33 |
| 4 | 95 | 83 |
| 8 | 96.5 | 87 |
| 12 | 96.5 | 92 |

1. A pharmaceutical composition comprising galantamine or a salt thereof, a portion of the contained galantamine being present in an immediate release form and another portion being present in an extended release form.

2. The pharmaceutical composition of claim 1, wherein portions of contained galantamine are present in more than one extended release form.

3. The pharmaceutical composition of claim 1, wherein an immediate release form comprises pharmacologically inert particles that are coated with galantamine or a salt thereof.

4. The pharmaceutical composition of claim 1, wherein an extended release form comprises pharmacologically inert particles that are coated with galantamine or a salt thereof, and having an outer coating comprising a rate controlling substance.

5. The pharmaceutical composition of claim 4, wherein a rate controlling substance comprises a cellulose polymer.

6. The pharmaceutical composition of claim 4, wherein a rate controlling substance comprises ethylcellulose.

7. A pharmaceutical composition comprising galantamine or a salt thereof in combination with a rate controlling substance, having an exterior coating of the same or a different rate controlling substance.

8. The pharmaceutical composition of claim 7, wherein a combination of galantamine or a salt thereof and a rate controlling substance is coated onto pharmacologically inert particles.

9. The pharmaceutical composition of claim 7, wherein a rate controlling substance comprises a cellulose polymer.

10. The pharmaceutical composition of claim 7, wherein a rate controlling substance comprises ethylcellulose.

11. A pharmaceutical composition comprising a capsule containing particles comprising galantamine or a salt thereof, the particles being coated with a rate controlling substance.

12. The pharmaceutical composition of claim 11, comprising a capsule containing pharmacologically inert particles having a coating comprising galantamine or a salt thereof and a rate controlling substance, and having an exterior coating comprising a rate controlling substance.

13. The pharmaceutical composition of claim 11, comprising a capsule containing tablets comprising galantamine or a salt thereof and having a coating comprising a rate controlling substance.

14. The pharmaceutical composition of claim 13, comprising a capsule containing particles comprising galantamine or a salt thereof in an immediate release form and particles comprising galantamine or a salt thereof in an extended release form.

15. The pharmaceutical composition of claim 11, wherein portions of contained galantamine are present in more than one extended release form.

16. The pharmaceutical composition of claim 11, wherein a rate controlling substance comprises a cellulose polymer.

17. The pharmaceutical composition of claim 11, wherein a rate controlling substance comprises ethylcellulose.
